(19) United States
(12) Patent Application Publication
(10) Pub. No.: US 2002/0138873 A1
(43) Pub. Date: Sep. 26, 2002

(54) Multiple component RNA vector system for expression of foreign sequences

(76) Inventors: Dennis J. Lewandowski, Auburndale, FL (US); William O. Dawson, Winter Haven, FL (US); Thomas H. Turpen, Vacaville, CA (US); Gregory P. Pogue, Vacaville, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/057,335

(22) Filed: Jan. 24, 2002

Related U.S. Application Data
(63) Continuation of application No. 09/265,575, filed on Mar. 9, 1999.

(51) Int. Cl. A01H 5/00; C12N 7/00

(52) U.S. Cl. 800/280; 435/235.1

(57) ABSTRACT

The present invention features a multiple component RNA vector system, which consists of RNA virus-derived RNA replicons and helper viruses. The present invention further features a method for producing foreign RNAs, effector RNAs, proteins or peptides in plants using the multiple component RNA vector system. Moreover, the present invention provides a method for stable and systemic production of foreign RNAs, effector RNAs, proteins and peptides using the multiple component RNA vector system.

The present application is a continuation of U.S. Ser. No. 09/265,575, filed Mar. 9, 1999, which is incorporated herein by references.

### FIELD OF THE INVENTION

The present invention relates generally to the field of RNA virus and plant genetics. More specifically, the present invention relates to a method for using replicating RNAs for production of foreign RNAs, effector RNAs, proteins or peptides in plants.

### BACKGROUND OF THE INVENTION

RNA viruses are a diverse family of infectious agents whose hosts include a wide variety of plants and animals. Their genomes consist of RNAs that replicate without forming a DNA intermediate and move from one host cell to another. The genome of an RNA virus can be composed of either one or multiple RNA segments. RNA viruses can be further divided into single-stranded RNA (ssRNA) or double-stranded RNA (dsRNA) viruses. SsRNA viruses can be further divided into positive-stranded, negative-stranded, or ambisense viruses. The genomic RNA of a positive-stranded RNA virus is messenger sense, which makes the naked RNA infectious.

Many plant viruses belong to the family of positive-stranded RNA viruses. They include tobacco mosaic tobamovirus (TMV), brome mosaic bromovirus (BMV), carnation mottle carmovirus (CarMV), and others. RNA plant viruses typically encode several common proteins, such as replicase/polymerase (non-structural) proteins essential for viral replication and mRNA synthesis, coat proteins providing protective shells for the extracellular passage, and other proteins required for the cell-to-cell movement, systemic infection and self-assembly of viruses.

Some RNA virus infections consist of a mixture of two kinds of RNAs: a replication-defective, helper-dependent RNA and a replication-competent virus. The replication-competent virus serves as a helper virus for the replication of the replication-defective RNA. There are primarily three types of replication-defective RNAs, replication-defective RNAs (dRNA) derived from the viral genomic RNA(s), satellite RNAs and satellite viruses, all of which replicate only in the presence of a helper virus. Satellite RNAs and viruses have genomes with little homology to their helper viruses (Francki, R., Ann. Rev. Microbiol. 39:151-174 (1985)). Some dRNAs negatively impact the replication of their helper virus and are thus termed defective interfering (DI) RNAs. DI RNAs are most frequently associated with high titer passage of animal viruses in cell culture (Holland, J., Virology, B. N. Fields and D. M. Knipe, Ed., 2^{nd} Ed., 1:151-165, Raven Press, New York (1990)). DI RNAs appear to evolve de novo, originating as simple deletion mutants that can acquire more complicated structures with additional replication cycles. Examples of viruses that produce DI RNAs include Sindbis virus and coronaviruses. Naturally occurring DI RNAs have been found in association with a few plant viruses that are members of the tombusvirus family, such as tomato bushy stunt virus (TBSV), turnip crinkle virus (TCV), and cymbidium ringspot virus (Hillman et al., Cell 51:427-433 (1987); Li et al., Proc. Natl. Acad. Sci. USA 86:9173-9177 (1989); and Burgyan et al., J. Gen. Virol. 70:235-239 (1989)). Also, dRNAs have been found associated with clover yellow mosaic potexvirus (White et al., J. Virol. 66:3069-3076 (1992)) and citrus tristeza closterovirus (Yang, et al., J. Gen. Virol. 78:1765-1769 (1997)) infections. Satellite RNAs, satellite viruses, and dRNAs require an essential function(s) provided by the helper virus in order to complete their replication cycle. The association of satellites or DI RNAs with a particular virus infection can lead to either an amelioration or enhancement of the pathological effect of the helper virus.

Both satellites and DI RNAs are often multiplied at the expense of the helper virus' own replication. Often, with repeated passages, the dominant pathogen RNA in an infected cell is that of the satellite RNA or DI RNA, with minimal helper virus RNAs detectable. Several researchers have exploited this property by expressing satellite RNAs in transgenic plants to confer resistance to disease caused by the helper virus (Gerlach et al., Nature 328:802-805 (1987); Harrison et al., Nature 328:799-802 (1987)). However this approach has not proven widely applicable since most plant viruses do not have naturally occurring satellite RNAs or DI RNAs associated with them, and also because these RNAs can often aggravate the severity of disease in unpredictable manners. To circumvent these problems, researchers have constructed dRNAs in vitro from the genomes of poliovirus (Hagino-Yamagishi et al., J. Virol. 63:5386-5392 (1989)) and BMV (Marsh et al., J. Gen. Virol. 72:1787-1792 (1991)). These virus-derived dRNAs or “RNA replicons” were artificially produced by deleting internal sequences from the normal autonomously replicating genomic RNAs of these viruses, which rendered them deficient in some essential character. These RNAs could no longer replicate autonomously, but required proteins supplied in trans by a co-inoculated helper virus. In the case of BMV, the RNA replicons were able to out-compete the co-inoculated helper virus for the replication machinery leading to dramatic reductions in the accumulation of the helper virus RNAs (Marsh et al., J. Gen. Virol. 72:1787-1792 (1991)). This competition was not dependent on any defective protein that may be translated from the RNA replicons, but was a consequence of the inherent replication advantage these smaller RNAs had over the larger helper RNA. When the RNA replicons were expressed in transgenic plants, these cells were effectively “resistant” to BMV infection (Marsh et al., J. Gen. Virol. 72:1787-1792 (1991)).

Construction of RNA replicons from full-length virus genomes is not a trivial matter. Many internal deletions introduced into virus genomes render the RNAs incapable of autonomous replication or from being replicated. Nevertheless, the presence of some non-replicating virus-derived RNA replicons may still be inhibitory to the multiplication of a co-inoculated wild type virus (Marsh et al., J. Gen. Virol. 72:2367-2374 (1991); Pogue et al., Virology 188:742-753 (1992)). Internal deletions may adversely affect the translation of essential proteins, debilitate the function of sequences within the same gene cluster (cis-acting sequences), or juxtapose RNA sequences in conformations inhibitory to replication. Once constructed, the replication competence of RNA replicons is unpredictable and often poor with many other necessary functions yet to be optimized, including packaging and competence for systemic movement in plants. The ability of an RNA replicon to be encapsidated and transported systemically in a plant along with the helper virus has been demonstrated for KL, a TMV-derived RNA replicon and TMV (Raffo and Dawson, Virology 184:277-289 (1991)). This RNA replicon, containing a deletion of a large portion of the non-structural coding region of TMV, replicated to reasonable levels in tobacco plants when co-inoculated with TMV. However, when isolated from systemic tissues, the KL replicon had undergone further evolution with a mixture of internal deletions identified in a variable population of RNAs. The successive replication cycles coupled with the constraints of packaging and systemic trafficking in plants appear to have pressured rapid evolution of the KL replicon.

### SUMMARY OF THE INVENTION

The present invention features a multiple component RNA vector system, which consists of one or more replication-competent helper viruses and one or more RNA replicons that are replicated by the helper virus in trans. The present invention further features a method for expressing one or more foreign RNAs, multiple effector RNAs, proteins or peptides in plants using the multiple component RNA vector system. Moreover, the present invention provides a method for stable and systemic expression of foreign RNAs, multiple effector RNAs, proteins or peptides using the multiple component RNA vector system.

In one embodiment, an RNA replicon may be engineered to contain a 5′ nontranslated region (NTR), an open reading frame (ORF) homologous to an ORF of the intact or fragments of a non-structural protein of an RNA virus, a sequence non-native to the RNA virus, and a 3′ NTR. The 3′ NTR of the RNA replicon may be native or non-native to the source of the 3′ NTR of a helper RNA virus. In addition, the 3′ NTR of the RNA replicon may be a hybrid of 3′ NTRs of two or more helper viruses. The 5′ NTR of the RNA replicon may also be native or non-native to the source of the 5′ NTR of the helper RNA virus. An RNA replicon may additionally contain, either from sources native to or non-native to the helper virus, one or more packaging signals, internal initiation sites, subgenomic mRNA promoter sequences, coat proteins, or movement proteins. An RNA replicon may also contain suitable restriction sites to facilitate the insertion of the non-native sequences. Preferably, the 5′ ORF of the RNA replicon encodes a sequence homologous to the intact or fragments of a non-structural protein of an RNA virus. An RNA replicon may be derived from a variety of RNA plant and animal viruses. In addition, an RNA replicon may be a hybrid, which contains native sequences from two or more viral sources.

In a second embodiment, a helper virus RNA may contain the wild type viral RNA sequence or its modified sequence to render a helper virus RNA more competent to replicate RNA replicons in trans. In addition, a hybrid helper RNA virus may be constructed to encompass native sequences from two or more RNA viral sources. Preferably, wild type tobacco mosaic virus and its mutated forms may be used in the present invention. Modifications of the wild type RNA plant virus may include the removal or the mutation of a suppressible stop codon, the removal or the replacement of an ORF for the coat protein, the replacement of the 3′ NTR, or the use of one or more subgenomic mRNA promoters, among others. The sequences inserted into modified forms of the wild type helper virus, such as those encoding for the coat protein, the 3′ NTR, or the subgenomic mRNA promoter, may be from a non-native source. Other modifications of the helper virus may include the modifications of RNA sequences in and/or near the suppressible stop codon to minimize the reversion to the wild type phenotype, for example the reversion of the sense codon to a stop codon. More preferably, TMV mutations that replace the suppressible stop codon may be used in the instant invention. Such TMV stop codon mutations may include tyrosine (TMV183Y), phenylalanine (TMV183F), serine (TMV183S), and the like. Most preferably, the TMV stop codon mutation, TMV183F, is particularly effective to function as a helper virus. A heterologous helper virus with respect to the RNA replicon may also be used to replicate RNA replicons. In particular, Odontoglossum ringspot virus (ORSV) as the helper virus is capable of replicating a variety of TMV RNA replicons. An RNA helper virus may also be derived from a variety of RNA animal viruses, such as poliovirus, alphaviruses, or rhinoviruses, among others. The helper viruses are complimentary to the RNA replicons in the multiple component vector system. The helper viruses may have one or more functional and structural proteins removed from the genome, which may prevent or disable the cell-to-cell movement of the helper viruses. The requisite functional and structural protein may be supplied in trans by the RNA replicons. These functional and structural proteins may include movement proteins, encapsidation proteins, among others. The reciprocal relationship between helper viruses and replicons may also be reflected in that the modified helper viruses may carry the foreign RNA, and/or produce multiple effector RNAs, proteins or peptides of interest in addition to their roles in facilitating the replication of RNA replicons.

In a third embodiment, the delivery of RNA replicons and suitable helper viruses into the plant may be effected by the inoculation of in vitro transcribed RNA, inoculation of virions, or internal inoculation of plant cells from nuclear cDNA, or the systemic infection resulting from any of these procedures. Any component of the vector system may be delivered by any of these procedures. In all cases, the co-infection may lead to a rapid and pervasive systemic expression of one or more foreign RNAs, effector RNAs, proteins or peptides in plant cells. The systemic infection of the plant by the RNA containing the protein gene(s) or sequences of interest may be followed by the growth of the infected host to produce the desired product, and the isolation and purification of the desired product, if necessary. The growth of the infected host is in accordance with conventional techniques, as is the isolation and the purification of the resultant product(s).

### BRIEF DESCRIPTION OF THE FIGURES

FIGS. 1A-1C are schematic diagrams of the various TMV and hybrid constructs described in this application. The ORFs for the TMV 126 kDa, 183 kDa, movement (mp) and coat (cp) proteins are indicated. ORSV sequences are indicated as shaded boxes. The ORFs for the wild type (gfp) and cycle 3 (c3gfp) green fluorescent proteins β-glucuronidase (gus) are indicated. Constructs containing a tyrosine (Y) or phenylalanine (F) substitution of the stop codon for the 126 kDa ORF are indicated. Sequences from the 3′-terninal portions of the TMV coat protein ORF are indicated as black boxes. 5′ and 3′ NTRs are indicated as solid lines. Coat protein subgenomic promoter (P) is indicated. FIG. 1 additionally contains schematic diagrams of some additional replicons that were used in experiments where Western blots were used to analyze expression. Construction was similar to that described in this application.

FIG. 2 shows the Western blot analysis of green fluorescent protein (GFP) expression resulting from the co-infection of Nicotiana sylvestris plants with RNA replicon TMV420 and helper virus TMV183F.

| Lane # | | (GFP) |
|---|---|---|
| 1. | purified GFP (15 ng) | +++ |
| 2. | BioRad High range MW markers | |
| 3. | Purified GFP (15 ng) | +++ |
| 4. | BioRad High range MW markers | |
| 5. | TMV183F + TMV420 N. tabacum non-inoculated | nd |
| | (upper leaf) | |
| 6. | TMV183F + TMV420 N. tabacum inoculated leaf | ++ |
| 7. | TMV183F + TMV420 N. sylvestris non-inoculated | nd |
| | (upper leaf) | |
| 8. | TMV183F + TMV420 N. sylvestris inoculated leaf | ++ |
| 9. | TMV183F + TMV420 N. tabacum inoculated leaf | ++ |
| 10. | Sigma prestained MW markers | |

nd = not detectable

FIG. 3 shows the Western blot of GFP expression in tobacco protoplasts from single component vector and multiple component RNA replicon vectors for the expression of foreign sequences.

| Lane # | | GFP |
|---|---|---|
| 1. | purified GFP (15 ng) | +++ |
| 2. | mock (water inoculated) protoplasts | — |
| 3. | TMV422 (single component vector) | ++ |
| | (1:5 dilution of lane 4) | |
| 4. | TMV422 (single component vector) | +++ |
| 5. | TMV004 + TMV420 (RNA replicon vector) | nd |
| 6. | TMV004 + TMV418 (RNA replicon vector) | + |
| 7. | TMV004 + TMV416 (RNA replicon vector) | + |
| 8. | TMV004 + TMV416 (RNA replicon vector) | nd |
| 9. | TMV004 | — |
| 10. | Sigma prestained MW markers | |

nd = not detectable

FIG. 4 shows Left: Northern blot analysis of the replication of TMV421 RNA replicon co-inoculated with helper virus TMV183F in Nicotiana tabacum plants using the TMV 3′ NTR-specific probe. Right: Northern blot analysis of the replication of TMV 421 RNA replicon co-inoculated with helper virus TMV183F in N. tabacum plants using the GFP-specific probe.

LEFT FIGURE

| Lane # | TMV 3′ NTR-specific probe |
|---|---|
| 1. | 183F N. tabacum (inoculated leaf) |
| 2. | 183F + TMV421 N. tabacum (inoculated leaf) |
| 3. | 183F + TMV421 N. tabacum (inoculated leaf) |

RIGHT FIGURE

| Lane # | GFP-specific probe |
|---|---|
| 1. | 183F N. tabacum (inoculated leaf) |
| 2. | 183F + TMV421 N. tabacum (inoculated leaf) |
| 3. | 183F + TMV421 N. tabacum (inoculated leaf) |

FIG. 5 shows the Northern blot analysis of the replication of TMV-based RNA replicons by the heterologous helper virus Odontoglossum ringspot virus (ORSV) using the TMV 3′ NTR-specific probe.

| Lane # | TMV 3′ NTR-specific probe |
|---|---|
| 1. | ORSV vRNA |
| 2. | ORSV vRNA + TMV420 (domain 1 and 2 RNA replicon + |
| | GFP) |
| 3. | ORSV vRNA + TMV408 (domain 1 RNA replicon + GFP) |
| 4. | ORSV vRNA + ΔCla (domain 1 RNA replicon) |
| 5. | ORSV vRNA + ΔCla/152 (domain 1 RNA replicon with full |
| | subgenomic mRNA promoter) |
| 6. | ORSV vRNA + TMV142/152 (domain 1 and 2 RNA replicon |
| | with full subgenomic mRNA promoter) |

FIG. 6 shows the Northern blot analysis of the replication of an RNA replicon vector expressing GFP in protoplasts co-inoculated with wild type TMV and different TMV-derived helper viruses using the TMV 3′ NTR-specific probe.

| Lane # | TMV 3′ NTR-specific probe |
|---|---|
| 1. | Wild type TMV + ΔCla/152/C3O3 |
| 2. | TMV183Y + ΔCla/152/C3O3 |
| 3. | TMV141 + ΔCla/152/C3O3 |
| 4. | TMV141Y + ΔCla/152/C3O3 |
| 5. | TMV163 + ΔCla/152/C3O3 |
| 6. | TMV163Y + ΔCla/152/C3O3 |
| 7. | TMV141/150 + ΔCla/152/C3O3 |
| 8. | TMV141/151 + ΔCla/152/C3O3 |
| 9. | TMV141/152 + ΔCla/152/C3O3 |
| 10. | S3-28 + ΔCla/152/C3O3 |
| 11. | TMV-CPO + ΔCla/152/C3O3 |
| 12. | TB2-GUS + ΔCla/152/C3O3 |

FIG. 7 shows the Northern blot analysis of the replication of an RNA replicon vector expressing GFP in protoplasts co-inoculated with wild type TMV and different TMV-derived helper viruses using the GFP-specific probe.

| In Lane # | GFP-specific probe |
|---|---|
| 1. | Wild type TMV + ΔCla/152/C3O3 |
| 2. | TMV183Y + ΔCla/152/C3O3 |
| 3. | TMV141 + ΔCla/152/C3O3 |
| 4. | TMV141Y + ΔCla/152/C3O3 |
| 5. | TMV163 + ΔCla/152/C3O3 |
| 6. | TMV163Y + ΔCla/152/C3O3 |
| 7. | TMY141/150 + ΔCla/152/C3O3 |
| 8. | TMV141/151 + ΔCla/152/C3O3 |
| 9. | TMV141/152 + ΔCla/152/C3O3 |
| 10. | S3-28 + ΔCla/152/C3O3 |
| 11. | TMV-CPO + ΔCla/152/C3O3 |
| 12 | TB2-GUS + ΔCla/152/C3O3 |

### DETAILED DESCRIPTION OF THE INVENTION

The present invention features a multiple component RNA vector system, which consists of one or more RNA replicons that are replicated by one or more suitable helper viruses. The present invention further features a method for producing foreign RNAs, multiple effector RNAs, proteins, or peptides in plants using the multiple component RNA vector system. Moreover, the present invention provides a method for stable and systemic production of foreign RNAs, multiple effector RNAs, proteins, or peptides using the multiple component RNA vector system.

In one embodiment, an RNA replicon may be engineered to contain a 5′ NTR, an ORF homologous to an ORF of the intact or fragments of a non-structural protein of an RNA virus, a sequence non-native to the RNA virus, and a 3′ NTR. The 3′ NTR in the RNA replicon may be native or non-native to the source of the 3′ NTR of a helper RNA virus. In addition, the 3′ NTR of the RNA replicon may be a hybrid of 3′ NTRs of two or more helper viruses. The 5′ NTR in the RNA replicon may also be native or non-native to the source of the 5′ NTR of the helper RNA virus. An RNA replicon may additionally contain, either from sources native to or non-native to the helper virus, one or more packaging signals, internal initiation sites, subgenomic mRNA promoter sequences, coat proteins, or movement proteins. An RNA replicon may also contain suitable restriction sites to facilitate the insertion of the non-native sequences. Preferably, the 5′ ORF of the RNA replicon encodes a sequence homologous to the intact or fragments of a non-structural protein of an RNA virus. An RNA replicon may be derived from a variety of RNA plant viruses, such as a potyvirus, a tobamovirus, a bromovirus, a carmovirus, a potexvirus, a closterovirus, a hordeivirus, a comovirus, alfalfa mosaic virus, or a bymovirus, among others. An RNA replicon may also be derived from a variety of RNA animal viruses, such as an alphavirus, poliovirus, or a rhinovirus, among others. An RNA replicon may contain sequences from a single virus, but may contain sequences from more than one virus, including viruses from more than one taxonomic group. A person skilled in the art will be able to construct an RNA replicon based on these viruses using the present invention.

In a preferred embodiment, tobacco mosaic virus (TMV) is used as the genetic backbone for RNA replicon construction. TMV produces two non-structural proteins, a 126 kDa protein (domains 1 and 2) and a 183 kDa protein (domains 1, 2 and 3) of the putative replicase. A TMV-derived RNA replicon for protein expression and RNA production at a minimum contains from the 5′ end to the 3′ end, a 5′ NTR native to the 5′ NTR of TMV, an ORF homologous to portions of a TMV nonstructural protein gene, a subgenomic mRNA promoter native or non-native to TMV, an non-native sequence, and a 3′ NTR native to or non-native to TMV. More preferably, a TMV-derived replicon may contain a 5′ NTR native to the 5′ NTR of TMV, nucleotide sequences encoding the intact or fragments of domains 1 and 2 of the TMV 126 kDa nonstructural protein, a subgenomic mRNA promoter, a sequence encoding the RNA or protein of interest, and a 3′ NTR native or non-native to that of a helper virus. The subgenomic mRNA promoter may be native to or non-native to TMV. For example, the subgenomic mRNA promoter for the TMV coat protein may be utilized. The 3′ NTR may be native to or non-native to TMV. For example, the 3′ NTR of the RNA replicon may be from the 3′ NTR of wild type TMV or from another tobamovirus. In addition, sequences encoding the intact or fragments of the TMV coat protein ORF may also be included in the genome of the TMV RNA replicon. For example, approximately 100, 200, or 300 nucleotides from the 3′ terminus of the TMV coat protein may be inserted between the end of a foreign sequence of interest and the 3′ NTR. For the construction of viable RNA replicons, inhibitory regions from the genomic RNA are typically removed. In TMV-derived RNA replicons, these inhibitory regions may include sequences encoding portions of the TMV 126 kDa protein from the 3′ terminus, portions of the movement protein, portions of the coat protein, or the readthrough portions of the TMV 183 kDa protein, among others.

In a second embodiment, a helper virus RNA may contain the wild type viral RNA sequence or its modified sequence to render a helper virus RNA more competent to replicate RNA replicons in trans. A helper RNA plant virus may be derived from a number of suitable RNA plant viruses, such as a potyvirus, a tobamovirus, a bromovirus, a carmovirus, a potexvirus, a closterovirus, a hordeivirus, a comovirus, alfalfa mosaic virus, or a bymovirus, among others. In addition, a hybrid helper RNA virus may be constructed to encompass native sequences from two or more RNA viral sources. Preferably, wild type tobacco mosaic virus and its mutated forms may be used in the present invention. Modifications of the wild type RNA plant virus may include the removal or the mutation of a suppressible stop codon, the removal or the replacement of an ORF for the coat protein, the replacement of the 3 ′ NTR, or the use of one or more subgenomic mRNA promoters, among others. The sequences inserted into modified forms of the wild type helper virus, such as those encoding for the coat protein, the 3′ NTR, or the subgenomic mRNA promoter, may be from a non-native source. Other modifications of the helper virus may include the modifications of RNA sequences in and/or near the suppressible stop codon to minimize the reversion to the wild type phenotype, for example the reversion of the sense codon to a stop codon. Examples of modifying the flanking sequences of the suppressible stop codon may be found in Skuzeski et al., J. Mol. Biol. 218:365-373 (1991). More preferably, TMV mutations that replace the suppressible stop codon may be used in the instant invention. Such TMV stop codon mutations may include tyrosine (TMV183Y), phenylalanine (TMV183F), serine (TMV183S), and the like. Most preferably, the TMV stop codon mutation, TMV183F, is particularly effective to function as a helper virus. A heterologous helper virus with respect to the RNA replicon may also be used to replicate RNA replicons. In particular, Odontoglossum ringspot virus (ORSV) as the helper virus is capable of replicating a variety of TMV RNA replicons. An RNA helper virus may also be derived from a variety of RNA animal viruses, such as poliovirus, alphaviruses, or rhinoviruses, among others. The helper viruses are complimentary to the RNA replicons in the multiple component vector system. The helper viruses may have one or more functional and structural proteins removed from the genome, which may prevent or disable the cell-to-cell movement of the helper viruses. The requisite functional and structural protein may be supplied in trans by the RNA replicons. These functional and structural proteins may include movement proteins, encapsidation proteins, among others. The reciprocal relationship between helper viruses and replicons may also be reflected in that the modified helper viruses may carry the foreign RNA, and/or produce multiple effector RNAs, proteins or peptides of interest in addition to their roles in facilitating the replication of RNA replicons.

In a third embodiment, the delivery of RNA replicons and suitable helper viruses into the plant may be affected by the inoculation of in vitro transcribed RNA, inoculation of virions, or internal inoculation of plant cells from nuclear cDNA, or the systemic infection resulting from any of these procedures. Any component of the vector system may be delivered by any of these procedures. In all cases, the co-infection may lead to a rapid and pervasive systemic expression of one or more foreign RNAs, multiple effector RNA, proteins or peptides in plant cells. The systemic infection of the plant by the RNA and protein gene(s) or sequences of interest may be followed by the growth of the infected host to produce the desired product, and the isolation and purification of the desired product, if necessary. The growth of the infected host is in accordance with conventional techniques, as is the isolation and the purification of the resultant product(s).

The foreign RNAs, effector RNAs, proteins or peptides produced in plant cells using the multiple component RNA vector system may be used to improve suitable traits in plants. Useful phenotypic traits in plant cells include, but are not limited to, improved tolerance to herbicides, improved tolerance to extremes of heat or cold, drought, salinity or osmotic stress, improved resistance to pests (insects, nematodes or arachnids) or diseases (fungal, bacterial or viral), production of enzymes or secondary metabolites, male or female sterility, dwarfness, early maturity, improved yield, vigor, heterosis, nutritional qualities, flavor or processing properties, prevention or inhibition of root development in malting barley, and the like. The production of proteins or peptides may also be directed to commercial use, such products include enzymes, antibodies, hormones, pharmaceuticals, melanins, vaccines, pigments, antibiotics, and the like. The multi-gene expression vectors contemplated in the multiple component RNA vector system may be particularly effective in expressing genes to manipulate multiple steps in a biosynthetic pathway, an antisense sequence to suppress endogenous expression of an unwanted plant gene, a gene for a product normally adversely affected by the “suppressed” plant gene, or a gene encoding a multi-hetermeric protein to be folded and fully assembled in vivo.

The recombinant RNA replicons and helper RNA viruses for the production of foreign sequences in plants are constructed using techniques well known in the art. Suitable techniques have been described in U.S. Pat. Nos. 5,316,931, 5,811,653, 5,866,785, 5,589,367, and 5,889,190, all incorporated herein by reference.

The multiple component RNA vector system disclosed in the present invention represents an improvement of a commercially used virus-based vector system that is being used to produce RNAs and proteins in plants. The commercially used system is typically a single-component vector into which a non-viral sequence is inserted and the resulting modified virus is used to infect plants. The improvement in the present invention allows for the production of multiple proteins or RNAs and allows for a more stable production of proteins that are unstable in the single-component vector. More specifically, the present invention features a stable multiple component RNA vector system that is capable of amplifying and expressing foreign proteins, RNAs or effector RNAs at high levels in plant cells. Many aspects of the multiple component RNA vector system for expression of RNAs and proteins are advantageous in providing flexibility and stability in the expression of foreign RNAs and proteins in plant cells. First, the reduced RNA replicon sizes in the multiple component vector system may render the RNA replicons in the instant invention able to accommodate larger sequences of foreign RNA or effector RNA, or sequences encoding proteins or peptides relative to the existing single component vectors. Virions from smaller RNA replicons may also facilitate more facile spreading through the plant. In addition, the smaller sizes of replicons may accommodate multiple subgenomic mRNA promoter or foreign gene cassettes in the same RNA. Size constraints are typically not applicable to the RNA replicons in the instant invention, until they contain very long sequences, typically up to 4 kb, of the additive foreign gene sequences. Second, the RNA replicons may be supplied by “internal inoculation” in the plant, for example from a nuclear gene, which will be amplified in each individual cell as a helper virus infects. The genetic stability of such a production system may be very high due to the fact that in each cell, the infection may be “re-inoculated” with a fresh RNA replicon derived from nuclear transcription. Third, the complementary relationship between the helper virus and RNA replicon is also advantageous. In this manner, the multiple component vector is mutually-complementing. The RNA replicon may carry one or more sequences which were removed from the helper virus, without which the movement of the helper virus from one cell to another is inhibited or disabled. Examples of these sequences are movement protein sequences, encapsidation sequences and other functional and structural sequences of any animal virus or plant virus. The foreign RNAs, effector RNAs, proteins or peptides of interest may be contained within either the replicon or the helper virus. And last, the RNA replicon expression vector may function with a single subgenomic mRNA promoter, two or more subgenomic mRNA promoters, including combinations of homologous and non-native subgenomic mRNA promoters, or a combination of subgenomic mRNA promoter(s) and internal ribosome initiation sites.

In order to provide a clear and consistent understanding of the specification and the claims, including the scope given herein to such terms, the following definitions are given:

5′ or 3′ NTR: nontranslated region of a viral genome at the 5′ or 3′ end, typically longer than 25 nucleotides and shorter than 500 nucleotides.

Cis-acting (cis-dependent): interaction of a molecule or complex with itself or between a gene product with the nucleic acid from which it was expressed.

Coat protein (capsid protein): an outer structural protein of a virus.

Effector RNA: RNA designed to cause a change in the host, such as gene silencing or regulation of gene expression.

Gene: a discrete nucleic acid sequence responsible for a discrete cellular product.

Helper virus: an arrangement of RNA sequences that facilitate the replication of itself and RNA replicons, when introduced into a cell of a host.

Homologous: nucleotide sequences that are substantially functionally equivalent to one another. Nucleotide differences between such sequences having substantial sequence homology will be de minimus in affecting function of the gene products or an RNA coded for by such sequence.

Host: a cell, tissue or organism capable of replicating a vector or viral nucleic acid and which is capable of being infected by a virus containing the viral vector or viral nucleic acid. This term is intended to include prokaryotic and eukaryotic cells, organs, tissues or organisms, where appropriate.

Infection: the ability of a virus to transfer its nucleic acid to a host or introduce viral nucleic acid into a host, wherein the viral nucleic acid is replicated, viral proteins are synthesized, and new viral particles assembled.

Internal initiation site: any of the internal regions that direct ribosome-mediated translation of mRNA into polypeptides.

Movement protein: a noncapsid protein required for cell-to-cell movement of RNA replicons or viruses in plants.

Non-native (foreign): any sequence that does not naturally occur in the virus or organism in which the sequence is said to be non-native (foreign).

Open Reading Framne: a nucleotide sequence of suitable length in which there are no stop codons.

Packaging signal: the RNA sequence(s) responsible for enclosing the RNA within the capsid or coat protein(s) to form a mature virion.

Plant Cell: the structural and physiological unit of plants, consisting of a protoplast and the cell wall.

Plant Tissue: any tissue of a plant in planta or in culture. This term is intended to include a whole plant, plant cell, plant organ, protoplast, cell culture, or any group of plant cells organized into a structural and functional unit.

Promoter: the 5′-flanking, non-coding sequence adjacent to a coding sequence which is involved in the initiation of transcription of the coding sequence.

Protoplast: an isolated cell without cell walls, having the potency for regeneration into cell culture or a whole host.

RNA replicon: an arrangement of RNA sequences generated by transcription of one or more non-native sequences that is capable of replication in the presence of a suitable helper virus when both are present in the same cell of a host. An RNA replicon may require sequences in addition to the replication origins for efficient replication and stability.

Subgenomic mRNA promoter: a promoter that directs the synthesis of an mRNA smaller than the full-length genome in size.

Trans-acting: interaction of a molecule or complex on other molecule(s) independent from itself or independent from the nucleic acid from which it was expressed.

Vector: a self-replicating RNA molecule that contains non-native sequences and which transfers an RNA segment between cells.

Virion: a particle composed of viral RNA, viral coat protein (or capsid protein).

Virus: an infectious agent composed of a nucleic acid encapsulated in a protein.

### EXAMPLES

The following examples further illustrate the present invention. These examples are intended merely to be illustrative of the present invention and are not to be construed as being limiting. The examples are intended specifically to illustrate recoveries of RNAs, proteins or peptides of interest which may be attained using the process within the scope of the present invention.

### Example 1

Identification of Non-Structural Protein Required for Viral Replication

Tobacco mosaic virus (TMV) is a positive-stranded ssRNA virus whose genome is 6395 nucleotides long. The genomic RNA contains a short 5′ NTR followed by an open reading frame (ORF) of 4848 nucleotides, which includes an amber stop codon at nucleotide 3417. Two non-structural proteins are expressed from this ORF. The first is a 126 kDa protein containing the nucleotide binding and putative helicase activities. The second is a 183 kDa protein, which is a translational readthrough of the amber stop codon in about 5-10% of the translational events. The 183 kDa protein contains the functional domains of the 126 kDa protein and a novel domain with homology to RNA-dependent RNA polymerases. At least two subgenomic mRNAs with a common 3′ terminus are also produced after TMV infection. These encode a 30 kDa movement protein and a 17.5 kDa coat protein. The 3′ terminus of TMV genomic RNA can be folded into a series of pseudoknots followed by a tRNA-like structure.

Experiments were conducted to examine whether the 126 kDa and/or the 183 kDa protein were essential for the replication of TMV. Standard molecular biological methods were used to introduce various mutations into the infectious TMV cDNA clones. The contributions of the two non-structural proteins to TMV replication were initially analyzed by transcribing the wild type or mutant cDNAs in vitro using T7 RNA polymerase and inoculating the transcripts into tobacco protoplasts. Plant cells were harvested at various time intervals post inoculation. RNA was subsequently extracted and analyzed by Northern blotting procedures. Viral replication in protoplasts separately infected with each virus was monitored by comparing the accumulation of positive- and negative-stranded RNAs corresponding to the mutant viral RNAs with that of the wild type virus.

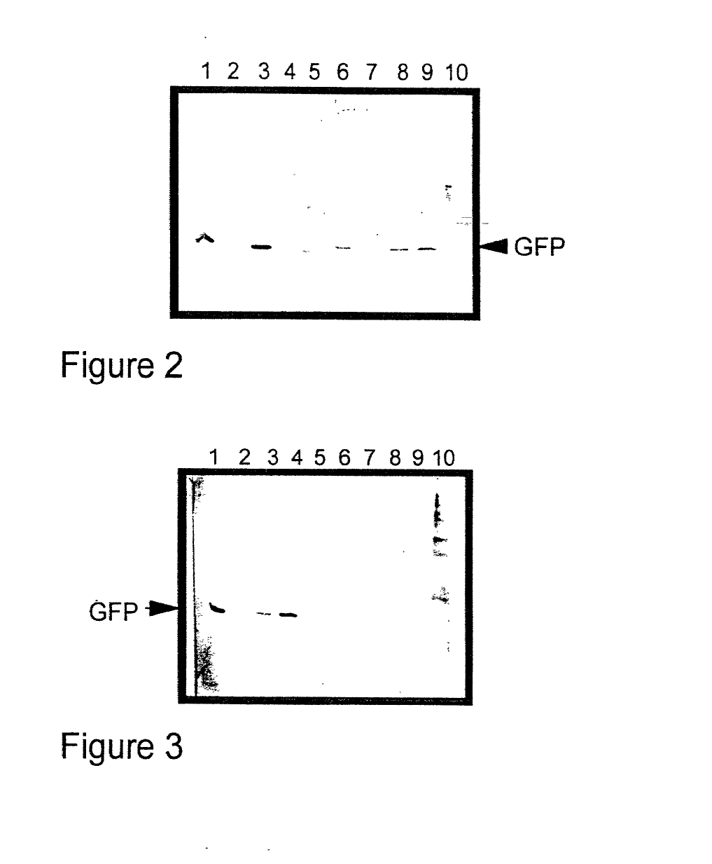

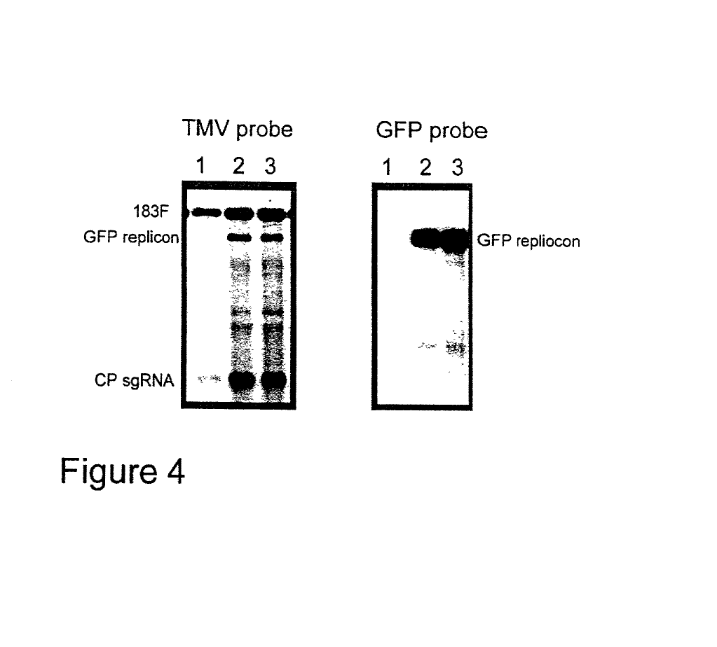

In one experiment, the suppressible stop codon was changed to a codon for tyrosine to produce a TMV mutant, TMV1 83Y. Protein immunoblotting confirmed that this TMV mutant produced only the 183 kDa protein with no accumulation of the 126 kDa protein. This TMV mutant was viable for the replication in plant cells, demonstrating that the 183 kDa protein of TMV is the functional viral replicase and is essential for the replication of TMV virus. Subsequent examples reveal that the 183 kDa protein is also trans-acting, which enables mutant TMV viruses expressing only the 183 kDa protein to function as helper viruses. In another experiment, the suppressible stop codon was changed to a codon for phenylalanine to produce another TMV mutant, TMV183F. Studies with the TMV183F mutant were consistent with the TMV183Y mutant in which the 183 kDa protein is trans-acting in enabling the replication of an RNA replicon. FIG. 2 shows the Western blot analysis of green fluorescent protein (GFP) expression resulting from the co-infection of Nicotiana sylvestris with the helper virus TMV183F and the RNA replicon TMV420. FIG. 3 shows the Western blot analysis of GFP expression resulting from co-infection of tobacco protoplasts with various RNA replicons and wild type TMV or by infection of tobacco protoplasts with single component vectors. FIG. 4 shows the Northern blot analysis of the replication of TMV421 RNA replicon co-inoculated with TMV183F as the helper virus in Nicotiana tabacum plants using the TMV 3′-specific probe (left) or using the GFP-specific probe (right).

Experiments also show that the 126 kDa protein is not required for the replication of viral RNAs. In addition, further experiments indicate that the 126 kDa protein serves an enhancing role in overall virus replication and functions in a cis-dependent manner.

### Example 2

Determination of the Optimal Sequence Arrangements in RNA Replicons

A. Internal Deletions

In order to construct RNA replicons capable of expressing foreign sequences in plants, a comprehensive deletion analysis of the TMV genome was carried out to identify suitable RNA replicons lacking the internal sequences, which would be replicated efficiently when co-inoculated with the wild type TMV as a helper virus. Standard molecular biological methods were employed to construct these deletion mutations in TMV cDNA clones. Following the successful construction, each mutant cDNA would be transcribed in vitro using T7 RNA polymerase and co-inoculated with in vitro transcripts derived from a wild type TMV cDNA into tobacco protoplasts. Plant cells were harvested at various time intervals post inoculation. RNA was subsequently extracted and analyzed by Northern blotting procedures. TMV replication was monitored by following the accumulation of positive- and negative-stranded RNAs corresponding to the mutant RNAs containing internal deletions and the wild type TMV RNA.

This deletion analysis revealed that some regions of the TMV genome could be removed without affecting the replication of the RNA replicon. Sequences encoding two thirds of the 126 kDa protein from its C-terminus, the entire 30 kDa movement protein and the coat protein of TMV were dispensable for replication and could be removed without preventing the replication of RNA replicons. Removal of sequences approximately encoding the readthrough portion of the 183 kDa protein was required for the detectable accumulation of RNA replicons in protoplasts. Unexpectedly, removal of some additional sequences in addition to the region approximately encoding the readthrough domain greatly increased the trans-replication of RNA replicons.

The deletion analysis also revealed that several internal regions are necessary for RNA replicons to replicate in the presence of a helper virus. The first region was the 5′ end of the genome, with the first approximately 256 nucleotides being essential and the first approximately 1342 nucleotides of the TMV genome providing the near optimal sequence length to be retained in the RNA replicon. In addition to the necessary 5′ NTR, replication of the RNA replicons also required that the 3′ NTR be present in the RNA replicon. The last feature necessary for efficient RNA replicon replication is the presence of an intact reading frame in the 5′ portion of the RNA replicon. Introduction of frame-shift mutations adversely affected the ability of RNA replicons to replicate efficiently.

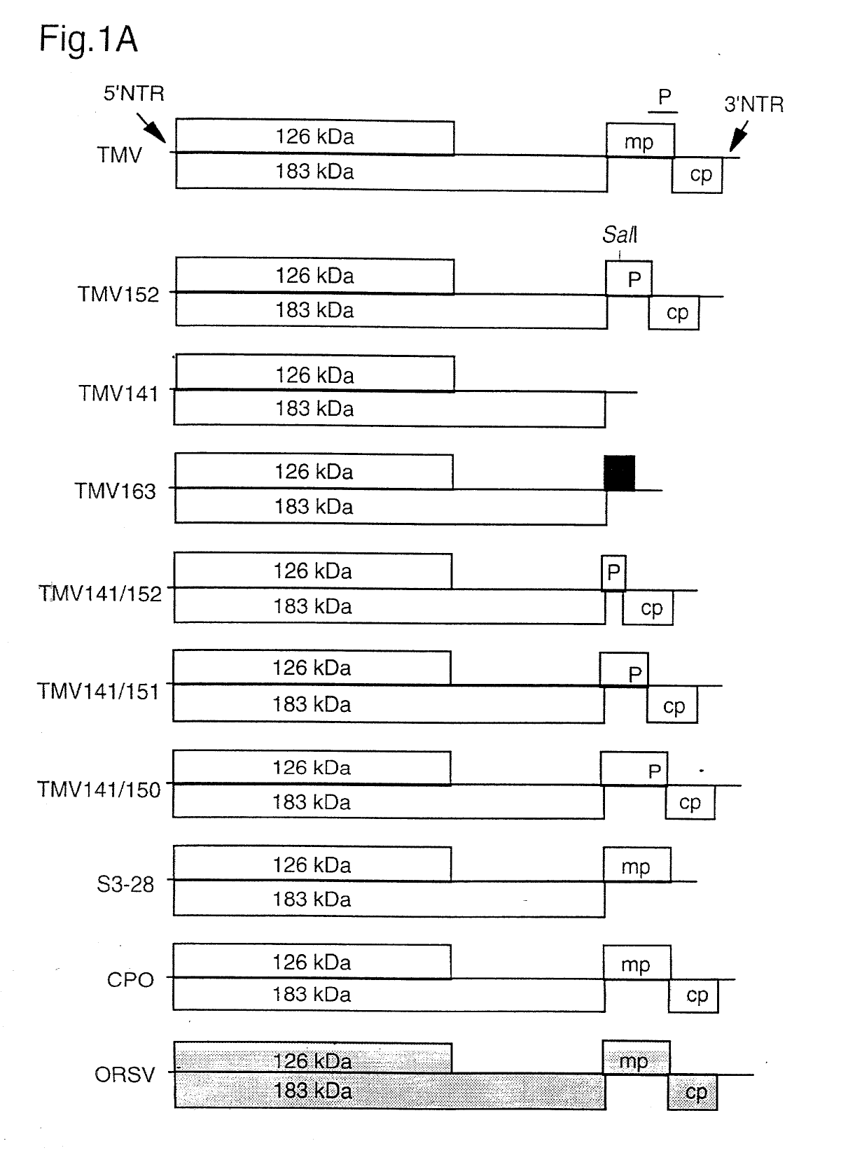

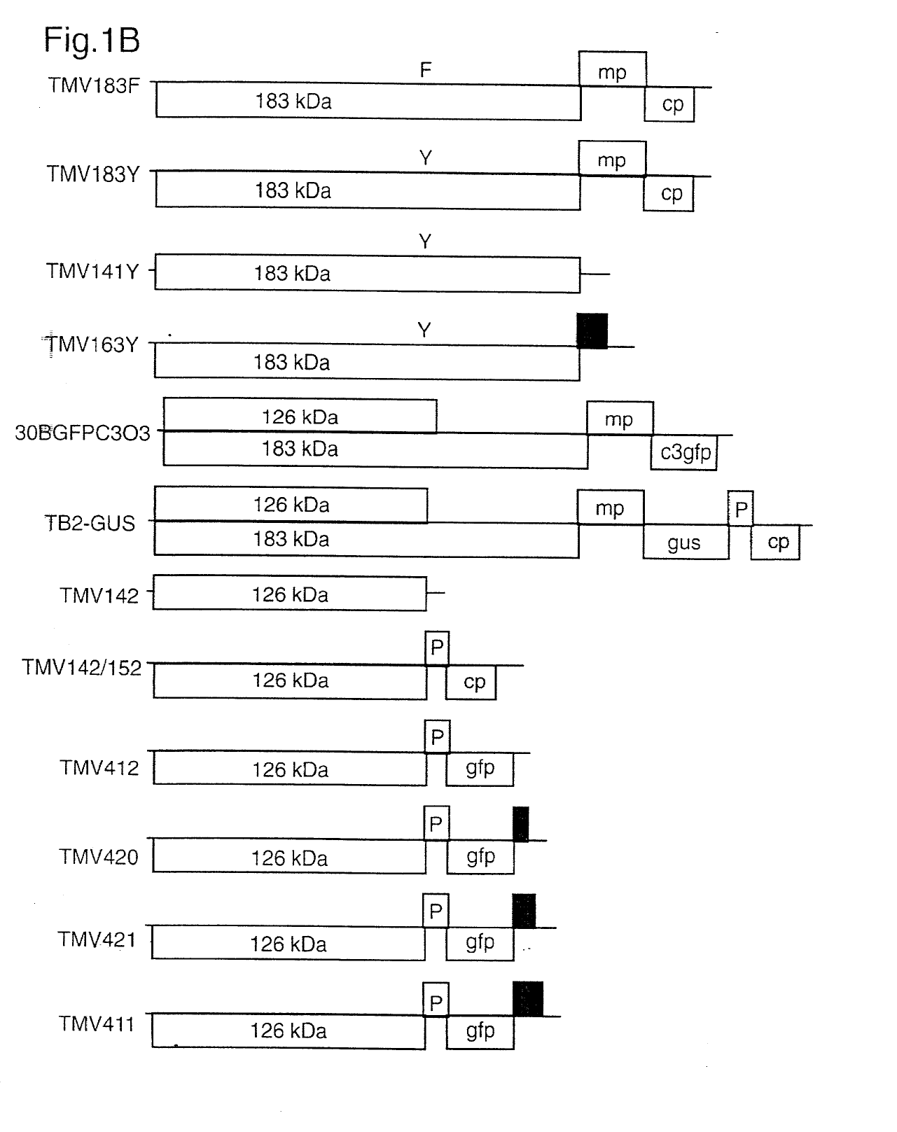

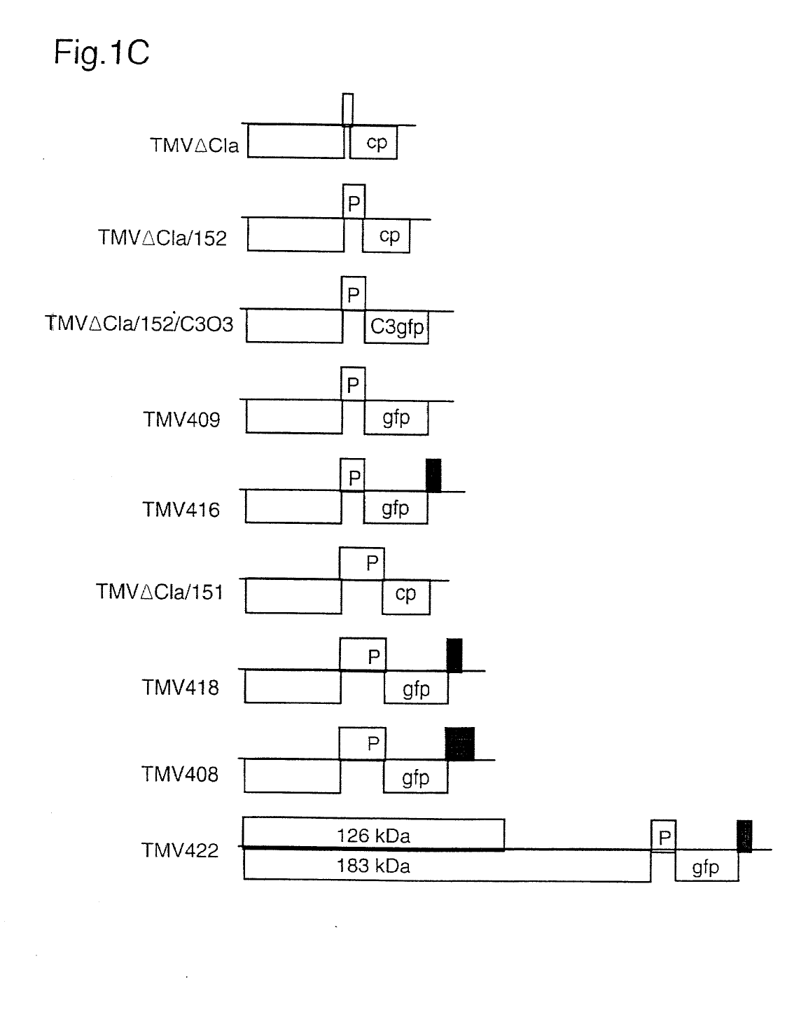

Comparison of the replication levels of the various functional RNA replicons derived from TMV revealed that the RNA replicon TMVΔCla conferred near maximal replication ability (FIG. 1). TMVΔCla contains the first 1342 nucleotides from the 5′ end of TMV and lacks sequences from this point to nucleotide position 5665 of the TMV genome. The 3′ end of this RNA replicon is contiguous with that of wild type TMV from nucleotide 5665 to the 3′ terminus (nucleotide 6395) of the RNA. This RNA often accumulates to levels similar to that of the genomic RNA of the wild type TMV helper virus. One negative characteristic of this RNA replicon is the lack of a complete subgenomic mRNA promoter sequence in the RNA replicon. An essential portion of the subgenomic mRNA promoter was deleted in the construction of this RNA replicon.

B. 3′ Terminal Sequences

The 3′ NTR of tobamoviruses includes an elaborately folded RNA structure with tRNA mimicking activities preceded by at least three RNA stem-loop regions that base pair with the adjacent sequences in a manner described as pseudoknot structures. Upstream of this set of pseudoknot RNA structures is the ORF encoding the TMV coat protein.

Comprehensive analysis of the 3′ end of the TMV genome was carried out with deletion of individual RNA structural elements in the viral CDNA, or by substitution of RNA elements from heterologous tobamoviruses into the viral CDNA (hybrid viruses). Standard molecular biological methods were employed to construct mutants in the TMV CDNA clone to analyze the contributions of each of these elements to the replication of the full-length TMV mutants. Following successful construction, a mutant CDNA containing intact ORFs for the 126 kDa and 183 kDa proteins would be transcribed in vitro using T7 RNA polymerase and transcripts would be inoculated into tobacco protoplasts. Plant cells were harvested at various time intervals post inoculation. RNA was subsequently extracted and analyzed by Northern blotting procedures. Effects on replication were monitored by comparing the accumulation of positive- and negative-stranded RNAs corresponding to the mutant viruses and hybrid viruses with that of the wild type virus. Hybrid RNA replicons containing the identical 3′ NTR substitutions were constructed, transcribed in vitro and co-inoculated into tobacco protoplasts with in vitro transcripts of the wild type TMV helper virus. Plant cells were harvested at various time intervals post inoculation. RNA was subsequently extracted and replication of the hybrid RNA replicons were analyzed by Northern blotting procedures. Replication of the hybrid RNA replicons was compared to replication of the unmodified TMV RNA replicon by measuring the accumulation of positive- and negative-stranded RNAs of the RNA replicons.

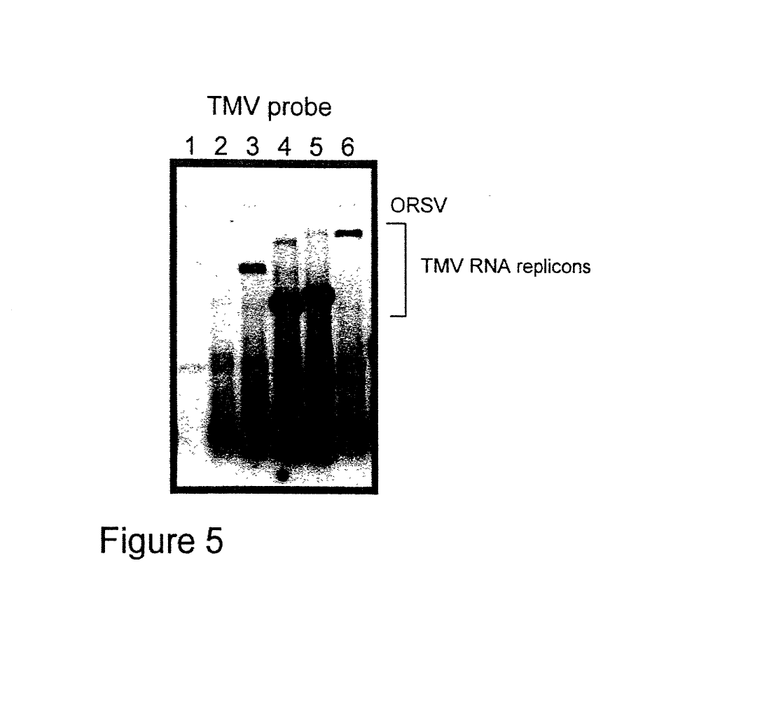

Full-length viral RNAs and RNA replicons containing the homologous TMV 3′ NTR accumulated to higher levels than RNAs containing a non-native (non-TMV) 3′ NTR. In addition, replication of some RNA replicons was supported by totally heterologous helper viruses. For example, some TMVΔCla-derived RNA replicons were replicated when co-inoculated with other heterologous helper viruses, such as ORSV. FIG. 5 shows the Northern blot analysis of the replication of TMV-based RNA replicons by the heterologous helper virus ORSV. Within the 3′ NTR, only the tRNA-like region and a single 3′ proximal pseudoknot structure is required for replication. The replication of some RNA replicons may be broadly supported by homologous, hybrid and also by completely heterologous helper viruses.

### Example 3

Development of an Expression Vector Using a TMV-based RNA Replicon as the Genetic Backbone

As noted in Example 2, the RNA replicon with the highest replication levels in the presence of wild type TMV is TMVΔCla. This dRNA however is incapable of high level expression of foreign genes due to the lack of a complete subgenomic mRNA promoter and the large amount of 5′ sequences in the vector which precludes the translation from the genomic RNA.

TMVΔCla was converted into expression vector TMVΔCla/152/C3O3 (see FIG. 1). Plasmid pTMVΔCla was first digested with the ClaI restriction enzyme and the sticky ends of the ClaI site were filled by the treatment with T4 DNA polymerase. The DNA was then digested with the KpnI restriction enzyme and the larger fragment containing the plasmid backbone and the 5′ 1343 nucleotides of the TMV genome was isolated. The 3′ one third of the TMV genome from TMV clone pTMV152 was digested with Sall. The SalI sticky ends were filled with T4 DNA polymerase, followed by digestion with KpnI restriction endonuclease. This smaller DNA fragment, which consists of a filled SalI site followed by TMV nucleotides 5460-6395 was ligated to the larger pTMVΔCla fragment to yield pTMVΔCla/152 (nucleotides 1344-5459 deleted). pTMVΔCla/152 was then digested with ClaI and KpnI and the larger DNA fragment was isolated. A ClaI/KpnI fragment from p30BGFPC3O3 was ligated into the larger fragment of pTMVΔCla/152. The resulting plasmid pTMVΔCla/152/C3O3 contains from the 5′ end to the 3′ end, the 5′ 1343 nucleotides from wild type TMV, a SalI restriction enzyme site, an intact TMV coat protein subgenomic mRNA promoter, a Pacd restriction site, the ORF for the cycle 3 green fluorescent protein (GFP), a XhoI restriction site, and the 3′ NTR from TMV (FIG. 1).

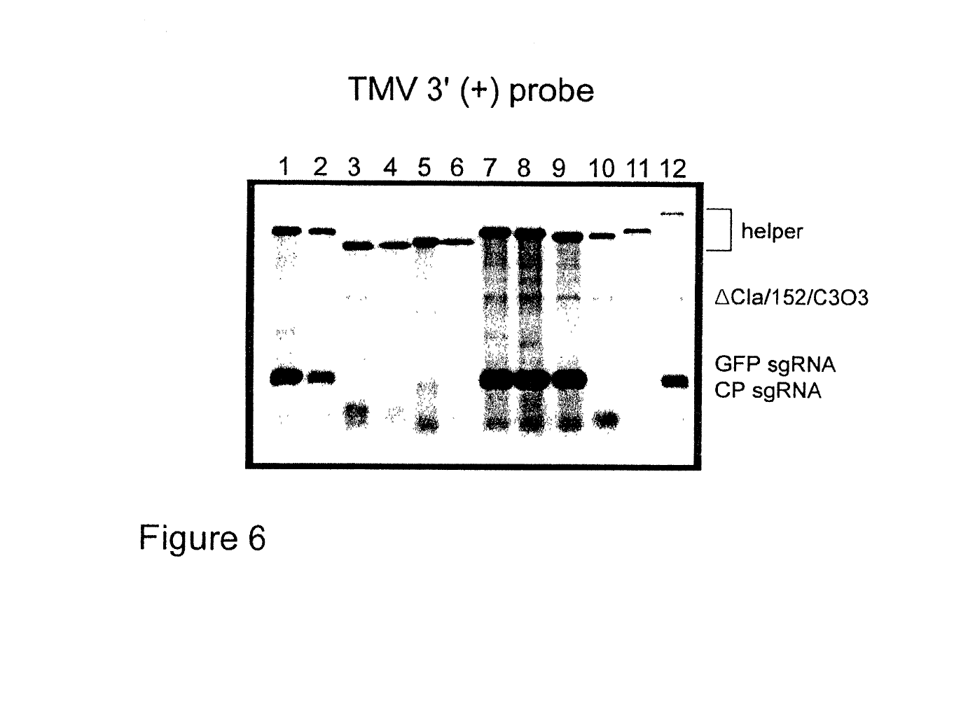

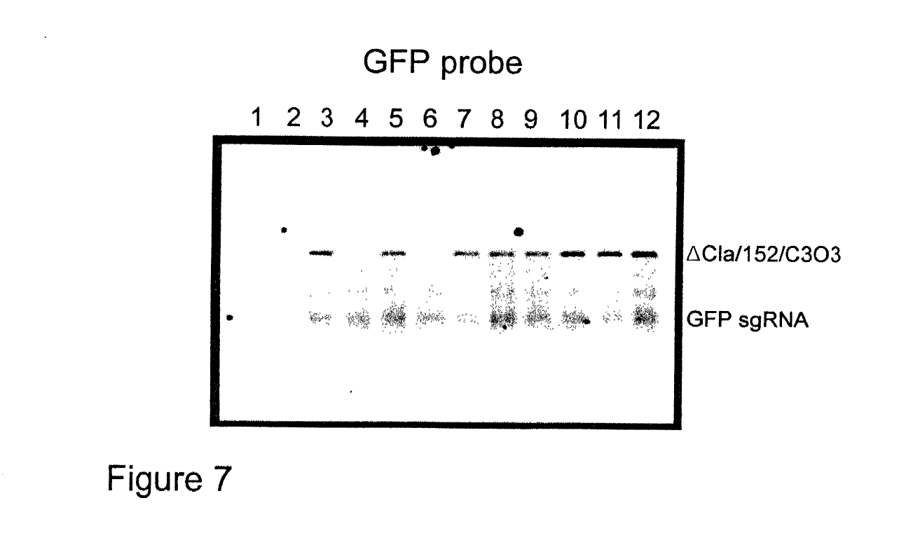

RNA transcripts were transcribed from KpnI-linearized pTMVΔCla/l 52/C3O3 and were co-inoculated with wild type TMV transcripts into tobacco protoplasts. Lane 1 in FIGS. 6 and 7 show the Northern blot analysis of the replication of TMVΔCla/152/C3O3 vector expressing GFP in protoplasts co-inoculated with wild type TMV using the TMV 3′ NTR-specific probe and the GFP-specific probe, respectively. The ΔCla/152/C3O3 RNA replicon was replicated and a subgenomic mRNA for GFP was detected, indicating that a functional RNA replicon-based vector had been constructed. This RNA replicon is a true expression vector due to the presence of a functional subgenomic mRNA promoter, restriction sites for insertion of foreign sequences and intact packaging signals for encapsidation with trans-supplied coat protein.

### Example 4

Modification of the Helper Virus to Enhance the Replication of RNA Replicons

In order to use the TMVΔCla/152/C3O3 RNA replicon as an expression tool, the replication of this RNA replicon must be maximized to ensure maximal levels of foreign gene expression. Several different TMV genomes were analyzed for their abilities to stimulate the replication of the TMVΔCla/152/C3O3 RNA replicon. Standard molecular biological methods were employed to construct TMV variants using cDNA clones for TMV and other tobamoviruses. Following the successful construction, a TMV variant to be tested as a helper virus would be transcribed in vitro using T7 RNA polymerase and transcripts co-inoculated with TMVΔCla/152/C3O3 transcripts into tobacco protoplasts. Plant cells were harvested at various time intervals post inoculations. RNA was subsequently extracted and analyzed by Northern blotting procedures. Replication of the TMV variants and the TMVΔCla/152/C3O3 RNA replicon were monitored by comparing the accumulation of positive- and negative-stranded RNAs. FIGS. 6 and 7 show the Northern blot analysis of the replication of TMVΔCla/152/C303 vector expressing GFP in protoplasts co-inoculated with different TMV-derived helper viruses using the TMV 3′ NTR-specific probe and the GFP-specific probe, respectively.

The general principle established from screening different TMV variants for the helper function was that the poorer the ability of the TMV variant to replicate independently, the better the helper virus it proved to be. This may be because the replication of the RNA replicons results from the excess replication capacity of the helper virus. If the helper virus utilizes its replicase efficiently, less synthetic machinery is available for replicating the RNA replicon. Interestingly, it was noted that the presence of the TMV RNA replicons did not appreciably reduce or inhibit the replication of any of the helper vinuses. This is contrary to results using BMV RNA replicons (Pogue et al., Virol. 178:152-160 (1990); Marsh et al., J. Gen. Virol. 72:2367-2374 (1991)). Experiments showed that TMV variants lacking the coat protein ORF (S3-28, FIG. 1), having the ORF of TMV coat protein replaced with that of the ORSV (TMV-CPO), or a dual subgenomic mRNA promoter vector (TB2-GUS), supported greater replication of the RNA replicon than did the wild type TMV (see FIGS. 1, 6 and 7).

TMV mutants, TMV183Y and TMV183F, that have the suppressible stop codon for the 183 kDa protein ORF modified, were able to amplify the TMVΔCla/152/C3O3 RNA replicon (see FIGS. 6 and 7). This result confirmed that the 183 kDa protein itself could function in trans for transcription as well as replication. Additional experiments showed that TMV183F was particularly effective as a helper virus. First, the TMV183F helper virus supported many dRNA-based RNA replicons revealed in the subsequent examples. Second, the TMV183F virus alone moved from cell to cell in tobacco, Nicotiana benthamiana, and N. sylvestris, which may lead to systemic infection of co-inoculated RNA replicons. Last, the TMV183F helper virus was more stable. This was evident in the studies of virus infection in planta when compared to the TMV183Y helper virus.

### Example 5

Multiple Component RNA Vector System for the Expression of GFP Reporter Protein Using a TMV-derived RNA Replicon Co-inoculated with TMV Helper Viruses

The ability of the TMVΔCla/152/C3O3 RNA replicon to express a functional GFP reporter gene in a plant cell was first assayed in tobacco protoplasts. RNA transcripts corresponding to TMVΔCla/152/C3O3 were co-inoculated with wild type TMV into tobacco protoplasts. Protoplasts were monitored for the accumulation of GFP protein with fluorescence microscopy at different time intervals post infection. Between 20-24 hours post inoculation, protoplasts viewed under UV illumination showed the characteristic green “glowing” phenotype, characteristic of accumulation of the GFP protein. The intensity of the green phenotype became greater during longer incubations of the protoplast cells. No such green color was observed in negative controls, which included healthy or “mock” (w,water inoculated) protoplasts or protoplasts infected with any of the helper viruses alone or the RNA replicon alone. Other helper virus constructs, including TMV183Y, TMVCPO, S3-28 and TB2-GUS, were also able to replicate and trans-activate the TMVΔCla/152/C3O3 RNA replicon to express the GFP protein in protoplasts, which demonstrates the robustness of the expression system. Between 10-30% of the infected protoplasts developed a significant green color which indicated that the GFP protein accumulation is dependent upon the replication of TMVΔCla/152/C3O3 and subsequent transcription and translation of the subgenomic mRNA encoding the GFP protein ORF. Northern blot analysis confirmed the presence of positive- and negative-stranded RNAs for the TMVΔCla/152/C3O3 RNA replicon and the accumulation of a subgenomic mRNA encoding the ORF sequence of the GFP protein.

### Example 6

Multiple Component RNA Vector System for the Expression of GFP Reporter Protein Using Other TMV-derived RNA Replicons Co-inoculated with TMV Helper Viruses

In the course of deleting sequences within the TMV genome to construct RNA replicons, experiments showed that an RNA replicon (TMV142) which had sequences downstream of the stop codon for the 126 kDa protein ORF up to the 3′ NTR deleted was unable to replicate on its own, but its replication could be supported by a replication competent helper virus, such as wild type TMV, a number of TMV mutants, and other heterologous helper viruses. In particular, this dRNA was more efficiently replicated by helper viruses that were unable to express the ORF of the TMV 126 kDa protein themselves (TMV183Y and TMV183F, see Example 4). Other modifications to the TMV142 RNA replicon that contained additional sequences from ORFs of the movement protein and/or coat protein were also constructed and were replicated to levels similar to TMV142 in protoplasts. This included the construction of pTMV142/152 which contained a coat protein subgenomic mRNA promoter and an ORF of the coat protein from the wild type TMV (see FIG. 1). This RNA replicon was constructed by digesting pTMV142 with the XhoI and KpnI restriction enzymes, which excised the 3′ NTR, and ligating the SalI/KpnI fragment containing the coat protein subgenomic mRNA promoter, coat protein and 3′ NTR from pTMV152 into TMV142. This plasmid gave rise to RNA replicons that were replicated in trans by various helper viruses. The coat protein subgenomic mRNA was also determined to be expressed from this RNA replicon.

To create an expression vector for expressing foreign sequences based on the genomic organization of the TMV142/152 RNA replicon, an intermediate GFP-expressing RNA replicon (TMV409), similar to pTMVΔCla/152/C3O3 in Example 5, was first constructed. Plasmid pTMVΔCla/152 was digested with ClaI and BsiWI. The larger fragment containing the plasmid backbone, 5′ TMV sequences up to the SalI site, subgenomic mRNA promoter sequences between SalI and ClaI, and 3′ NTR sequences between BsiWI and KpnI is retained. The ClaI/BsiWI fragment from pTMV30BGFP containing the remainder of the subgenomic mRNA promoter between the ClaI and PacI sites, the wild type ORF of the GFP protein, and the 3′ NTR sequences up to BsiWI, was ligated into SalI/BsiWI-digested pTMVΔCla/152 to create pTMV409. This resulted in an RNA replicon capable of expressing GFP upon co-infection with and replication by a suitable helper virus. To insert the subgenomic mRNA promoter and the ORF of the GFP protein into an RNA replicon encoding domains 1 and 2 of the non-structural proteins, pTMV409 was digested with SalI and KpnI. The resulting fragment containing the subgenomic mRNA promoter, the ORF of the GFP protein, and the 3′ NTR was ligated into AhoI/KpnI-digested pTMV142 to create pTMV412. The TMV412 RNA replicon was replicated in tobacco protoplasts and leaves of N. benthamiana co-inoculated with in vitro RNA transcripts of TMV412 and a suitable helper virus. TMV412 contains from the 5′ end to the 3′ end, the 5′ NTR from TMV, the ORF for the complete TMV 126 kDa protein, full TMV coat protein subgenomic mRNA promoter, PacI site, the wild type ORF of the GFP protein, XhoI sites, and the TMV 3′ NTR (see FIG. 1). It is an effective expression vector which features PacI and XhoI restriction enzyme sites for the insertion of foreign sequences and also the functional packaging signals for the encapsidation with coat protein supplied in trans by helper viruses.

At various times post inoculation, RNA and protein samples were taken from plants co-inoculated with a suitable helper virus and RNA replicon TMV412 and analyzed for the accumulation of viral RNAs and the accumulation of the GFP protein. Samples were also analyzed under UV illumination to visualize the infected areas. Northern blot analysis confirmed the presence of both the helper virus and the GFP-expressing TMV412 RNA replicon from the leaf tissue. It was determined that helper viruses that did not express their own 126 kDa protein, including TMV183F, were superior helper viruses for supporting replication of the TMV412 RNA replicon. GFP protein expression was confirmed by Western immmunoblot analysis from the co-infected tissues of N. benthamiana and N. tabacum. Other modifications include the insertion of additional 3′ proximal sequences from the ORF of the TMV coat protein between the XhoI site for foreign gene insertion and the 3′ NTR. It was determined that the inclusion of these sequences improved the accumulation levels of the RNA replicon and enhanced GFP expression levels (see subsequent examples).

### Example 7

Multiple Component RNA Vector System for Protein Expression Using TMV420, TMV421, and TMV411 RNA Replicons Co-inoculated With TMV Helper Viruses

Three additional RNA replicons, TMV420, TMV421, and TMV411, encoding domains 1 and 2 of the 183 kDa protein were constructed according to the method in Example 6. These three RNA replicons are analogous to the genetic organization of the TMV412 RNA replicon with the exception that each contained an insertion of a portion of the ORF of the TMV coat protein. TMV420 contains from the 5′ end to the 3′ end, the 5′ NTR, the ORF of a functional 126 kDa protein, full TMV coat protein subgenomic mRNA promoter, PacI site, an ORF of the GFP protein, XhoI site, 100 nucleotides of the TMV coat protein from the 3′ end, and the 3′ NTR (see FIG. 1). TMV421 and TMV411 contain identical constructions to TMV420 with the exception that these RNA replicons included 200 and 300 nucleotides of the TMV coat protein from the 3′ end, respectively (see FIG. 1). Replication of RNA replicons TMV420, TMV421 and TMV411 was confirmed in tobacco protoplasts and leaves of N. benthamiana and N. tabacum plants when co-inoculated with helper virus TMV183F. Successful amplification of each RNA replicon was demonstrated by Northern blot analysis. Expression of the GFP protein in inoculated leaves of N. benthamiana and N. tabacum was determined visually by UV illumination. Tn addition, the dRNA-based RNA replicon TMV420 was also tested in N. sylvestris by co-inoculation with the helper virus TMV183F. Replication of TMV420 was shown by Northern blot hybridization. The expression of the GFP protein in inoculated leaves of N. sylvestris were also determined visually by UV illumination and Western immunoblot analysis.

### Example 8

Systemic Expression of the GFP Reporter Protein Using a RNA Replicon Co-inoculated with a TMV Helper Virus

N. benthamiana plants co-inoculated with the TMV420 RNA replicon and the helper virus TMV183F showed systemic infection of plant cells with TMV183F and the TMV420 RNA replicon. N. benthamiana plants were inoculated with virions prepared from the lysate of tobacco protoplasts co-inoculated with in vitro transcripts of TMV183F and TMV420 following 20 hours of culture. Six to seven days post-inoculation, the fluorescence of the GFP reporter protein was detectable in the inoculated leaves with long wave WV illumination. Tissues that fluoresced green under UV light were further analyzed to confirm the presence of genomic RNAs from both the helper virus TMV183F and the RNA replicon TMV420 by Northern blotting. GFP protein expression was confirmed by Western immunoblot analysis. Some of the upper, non-inoculated, symptomatic leaves from plants co-inoculated with TMV183F and TMV420 developed areas that fluoresced green under long wave UV illumination. Although plants inoculated with TMV183F alone exhibited systemic symptoms identical to the plants co-inoculated with TMV183F and TMV420, none of the plants inoculated with TMV183F alone had any green fluorescence in either the inoculated or the upper systemically infected leaves.

### Example 9

Expression of Multiple Foreign Sequences Using RNA Replicons Co-inoculated with TMV Helper Viruses

To demonstrate the feasibility of viral-based multiple component vectors for expressing multiple RNAs or proteins, tobacco protoplasts were co-inoculated with TB2-GUS as the helper virus and TMVΔCla/152/C3O3 as the RNA replicon. TB2-GUS is a TMV-based dual subgeniomic mRNA promoter vector expressing the bacterial β-glucuronidase (GUS) gene, followed by a non-native extra subgenomic mRNA promoter and coat protein from ORSV. At 20 and 40 hpi, protoplasts were analyzed for the simultaneous expression of GFP and GUS proteins. The GFP expression was monitored under fluorescence microscopy to detect the green, GFP-expressing cells. To detect the expression of GUS, a parallel aliquot of the cells analyzed for the GFP expression were incubated in the presence of the substrate 5-bromo-4-chloro-3-indoyl-β-D-glucuronide at 37° C. Only samples from protoplasts inoculated with TB2-GUS and TMVΔCla/152/C3O3 were positive for both GFP and GUS expression. RNA samples were also taken at 20 hpi and analyzed by Northern blot hybridization. Northern blotting revealed the replication of both the helper virus TB2-GUS and the RNA replicon TMVΔCla/152/C3O3 genomic RNAs. Further, the GFP-specific hybridization probes revealed the presence of the GFP subgenomic RNA. The example further demonstrates that the foreign RNAs or proteins of interest may be included in not only the RNA replicon, but also in the helper virus, which provides greater flexibility and stability in expressing foreign RNAs or proteins in the multiple component RNA vector system.

While the invention has been disclosed in this patent application by reference to the details of preferred embodiments of the invention, it is to be understood that this disclosure is intended in an illustrative rather than limiting sense, as it is contemplated that modifications will readily occur to those skilled in the art, within the spirit of the invention and the scope of the appended claims.

We claim:

1. A system comprising:
one or more RNA replicons, wherein at least one RNA replicon comprises:
(1) a5′ NTR,
(2) an open reading frame homologous to an open reading frame of an intact, or one or more functional fragments of, non-structural protein from an RNA virus,
(3) at least one sequence non-native to said RNA virus, cell, and
(4) a 3′ NTR; and
wherein said RNA replicon is incapable of replicating self; and one or more helper RNA viruses wherein at least one helper virus facilitates the replication of said RNA replicon in a plant or a plant cell.

2. The system according to claim 1 wherein said 3′ NTR of said RNA replicon is native to the 3′ NTR of at least one helper RNA virus.

3. The system according to claim 1 wherein said 3′ NTR of said RNA replicon is non-native to the 3′ NTR of any said helper RNA viruses.

4. The system according to claim 1 wherein said 3′ NTR of said RNA replicon is a hybrid of the 3′ NTRs of two or more said helper RNA viruses.

5. The system according to claim 1 wherein said RNA replicon further comprises at least one subgenomic mRNA promoter.

6. The system according to claim 5 wherein said subgenomic mRNA promoter is native to at least one said RNA virus.

7. The system according to claim 6 wherein said native subgenomic mRNA promoter is a promoter for a coat protein of at least one said RNA virus.

8. The system according to claim 7 wherein said subgenomic mRNA promoter is non-native to any said RNA viruses.

9. The system according to claim 1 wherein said RNA replicon further comprises an open reading frame homologous to an open reading frame of an intact coat protein or a fragment thereof from at least one said RNA virus.

10. The system according to claim 1 wherein said RNA replicon further comprises a coat protein non-native to any said RNA viruses.

11. The system according to claim 1 wherein said RNA replicon further comprises an open reading frame homologous to an open reading frame of an intact movement protein or a fragment thereof from at least one said RNA virus.

12. The system according to claim 1 wherein said RNA replicon further comprises a movement protein non-native to any said RNA viruses.

13. The system according to claim 1 wherein said RNA replicon further comprises an origin of assembly.

14. The system according to claim 1 wherein said RNA replicon further comprises at least one internal ribosome initiation site.

15. The system according to claim 1 wherein at least one said RNA virus is a positive-stranded ssRNA virus.

16. The system according to claim 15 wherein said positive-stranded ssRNA is selected from the plant virus group consisting of potyviruses, tobamoviruses, bromoviruses, carnoviruses, potexviruses, closteroviruses, bymoviruses, and hordeiviruses.

17. The system according to claim 16 wherein said positive-stranded ssRNA virus is a tobacco mosaic virus.

18. The system according to claim 1 wherein said RNA replicon comprises:
(1) a 5′ NTR native to the 5′ NTR of a tobamovirus;
(2) an open reading frame homologous to an open reading frame of an intact non-structural protein or a fragment thereof from said tobamovirus;
(3) a subgenomic mRNA promoter;
(4) a sequence non-native to said tobamovirus; and
(5) a 3′ NTR native to the 3′ NTR of at least one helper RNA virus.

19. The system according to claim 18 wherein said RNA replicon comprises:
(1) a sequence homologous to about 1340 nucleotides at the 5′ end of a tobacco mosaic virus;
(2) a subgenomic mRiNA promoter;
(3) a sequence non-native to said tobacco mosaic virus; and
(4) a 3′ NTR native to the 3′ NTR of at least one helper RNA virus.

20. The system according to claim 18 wherein said RNA replicon comprises:
(1) a 5′ NTR native to the 5′ NTR of a tobacco mosaic virus;
(2) an open reading frame homologous to an open reading frame of an intact non-structural protein or a fragment thereof from said tobacco mosaic virus;
(3) a subgenomic mRNA promoter;
(4) a sequence non-native to said tobacco mosaic virus; and
(5) a 3′ NTR native to the 3′ NTR of at least one helper RNA virus.

21. The system according to claim 20 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgenomic mRNA promoter for a coat protein of said tobacco mosaic virus;
(4) a sequence non-native to said tobacco mosaic virus; and
(5) a 3′ NTR native to the 3′ NTR of at least one helper RNA virus.

22. The system according to claim 21 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgenomic mRNA promoter for a coat protein of said tobacco mosaic virus;
(4) a sequence non-native to tobacco mosaic virus;
(5) a sequence homologous to about 100 nucleotides from the 3′ end of an open reading frame of a coat protein of said tobacco mosaic virus; and
(6) a 3′ NTR native to the 3′ NTR of at least one helper RNA virus.

23. The system according to claim 21 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgenomic mRNA promoter for a coat protein of said tobacco mosaic virus;
(4) a sequence non-native to said tobacco mosaic virus;
(5) a sequence homologous to about 200 nucleotides from the 3′ end of an open reading frame of a coat protein of said tobacco mosaic virus; and
(6) a 3′ NTR native to the 3′ NTR of at least one helper RNA virus.

24. The system according to claim 21 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgenomic mRNA promoter for a coat protein of said tobacco mosaic virus;
(4) a sequence non-native to said tobacco mosaic virus;
(5) a sequence homologous to about 300 nucleotides from the 3′ end of an open reading frame of a coat protein of said tobacco mosaic virus; and
(6) a 3′ NTR native to the 3′ NTR of at least one helper RNA virus.

25. The system according to claim 1 wherein said RNA replicon comprises:
(1) a 5′ NTR native to the 5′ NTR of a tobamovirus;
(2) an open reading frame homologous to an open reading frame of an intact, or one or more fragments of, non-structural protein from said tobamovirus;
(3) a subgenomic mRNA promoter;
(4) a sequence non-native to said tobamovirus; and
(5) a 3′ NTR non-native to the 3′ NTR of any said helper RNA viruses.

26. The system according to claim 25 wherein said RNA replicon comprises:
(1) a sequence homologous to about 1340 nucleotides at the 5′ end of a tobacco mosaic virus;
(2) a subgenomic mRNA promoter;
(3) a sequence non-native to said tobacco mosaic virus; and
(4) a 3′ NTR non-native to the 3′ NTR of any said helper RNA viruses.

27. The system according to claim 25 wherein said RNA replicon comprises:
(1) a 5′ NTR native to the 5′ NTR of a tobacco mosaic virus;
(2) an open reading frame homologous to an open reading frame of an intact, or one or more fragments of, non-structural protein from said tobacco mosaic virus;
(3) a subgenomic mRNA promoter;
(4) a sequence non-native to said tobacco mosaic virus; and
(5) a 3′ NTR non-native to the 3′ NTR of any said helper RNA viruses.

28. The system according to claim 27 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter homologous to a subgenomic mRNA promoter for a coat protein of said tobacco mosaic virus;
(4) a sequence non-native to said tobacco mosaic virus; and
(5) a 3′ NTR non-native to the 3′ NTR of any said helper RNA viruses.

29. The system according to claim 28 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgcnomic mRNA promoter for a coat protein of said tobacco mosaic virus;
(4) a sequence non-native to said tobacco mosaic virus;
(5) a sequence homologous to about 100 nucleotides from the 3′ end of an open reading frame of a coat protein of said tobacco mosaic virus; and
(6) a 3′ NTR non-native to the 3′ NTR of any said helper RNA viruses.

30. The system according to claim 28 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic mRNA promoter native to a subgenomic mRNA promoter for a coat protein of said tobacco mosaic virus;
(4) a sequence non-native to said tobacco mosaic virus;
(5) a sequence homologous to about 200 nucleotides from the 3′ end of an open reading frame of a coat protein of said tobacco mosaic virus; and
(6) a 3′ NTR non-native to the 3′ NTR of any said helper RNA viruses.

31. The system according to claim 28 wherein said RNA replicon comprises:
(1) a 5′ NTR native to the 5′ NTR of a tobacco mosaic virus;
(2) an open reading frame of an intact 126 kDa non-structural protein of said tobacco mosaic virus;
(3) a subgenomic MnRNA promoter native to a subgenomic mRNA promoter for a coat protein of said tobacco mosaic virus;
(4) a sequence non-native to said tobacco mosaic virus;
(5) a sequence homologous to about 300 nucleotides from the 3′ end of an open reading frame of a coat protein of said tobacco mosaic virus; and
(6) a 3′ NTR non-native to the 3′ NTR of any said helper RNA viruses.

32. The system according to claim 1 wherein said helper RNA virus is obtained from a positive-stranded ssRNA virus.

33. The system according to claim 32, wherein said positive-stranded ssRNA virus is selected from the plant virus group consisting of potyviruses, tobamoviruses, bromoviruses, carmoviruses, potexviruses, closteroviruses, bymoviruses, and hordeiviruses.

34. The system according to claim 1 wherein at least one helper RNA virus and at least one RNA replicon are obtained from the same viral source.

35. The system according to claim 1 wherein any said helper RNA virus is non-native to any viral sequence in any said RNA replicon.

36. The system according to claim 1 wherein said helper RNA virus is modified to effect the replication of said RNA replicon more efficiently than the wild type of said helper RNA virus.

37. The system according to claim 1 wherein at least one helper RNA virus is modified to contain a sequence non-native to said helper RNA virus.

38. A method for producing an RNA or a protein in plants comprising the steps of:
(a) obtaining one or more RNA replicons derived from RNA viruses, wherein at least one RNA replicon comprises:
(1) a 5′ NTR,
(2) an open reading frame native to an open reading frame of an intact, or one or more fragments of, non-structural protein from a RNA virus,
(3) at least one sequence non-native to said RNA virus, and
(4) a 3′ NTR;
(b) obtaining one or more helper viruses wherein at least one helper virus effects the replication of said RNA replicon; and
(c) co-inoculating said one or more RNA replicons and said one or more helper RNA viruses.

39. A method according to claim 38 wherein at least one RNA replicon is delivered by internal inoculation.

40. A method according to claim 38 wherein at least one helper RNA virus is delivered by internal inoculation.

41. A host plant inoculated according to the method of claim 38.

42. A method for producing an RNA or a protein in plants comprising the steps of:
(a) obtaining one or more RNA replicons derived from RNA viruses, wherein at least one RNA replicon comprises:
(1) a 5′ NTR,
(2) an open reading frame native to an open reading frame of an intact, or one or more fragments of, non-structural protein from a RNA virus,
(3) at least one sequence non-native to said RNA virus, and
(4) a 3′ NTR;
(b) obtaining one or more helper RNA viruses wherein at least one helper RNA virus effects the replication of said RNA replicon and at least one helper RNA virus contains a sequence non-native to any said helper RNA viruses; and
(c) co-inoculating said one or more RNA replicons and said one or more helper RNA viruses.

43. A plant host containing the system of claim 1.
